(19) United States
(12) Patent Application Publication
(10) Pub. No.: US 2026/0033890 A1
(43) Pub. Date: Feb. 5, 2026

(54) Extractor Injector Introducer Kit (EIIK)

(71) Applicant: Rassan M Tarabein, Mobile, AL (US)

(72) Inventor: Rassan M Tarabein, Mobile, AL (US)

(21) Appl. No.: 19/342,972

(22) Filed: Sep. 29, 2025

(51) Int. Cl. A61B 18/14; A61B 18/00; A61B 90/00

(52) U.S. Cl. CPC A61B 18/1477; A61B 90/02; A61B 2018/00184; A61B 2018/00577; A61B 2018/1432

(57) ABSTRACT

The Extractor-Injector-Introducer Kit (EIIK) is a modular surgical system designed for minimally invasive extraction, injection, and instrument introduction to delicate tissue sites, including the brain. The kit includes a steerable radiofrequency (RF) needle (10), a sequence of concentric dilator tubes (20, 22, 24), an extractor tube (30) with a distal cutting element (32), and an injector tube (40) with a delivery lumen (42). The RF needle (10) may be coupled to an RF generator (14) for controlled ablation. The system provides safe, guided access with reduced trauma to surrounding tissue.

### BACKGROUND OF THE INVENTION

Access to delicate tissue structures, such as those in the brain and vital organs, remains a major surgical challenge. Existing tools often lack versatility, requiring multiple separate devices for extraction, injection, and introducer functions, thereby increasing trauma and operative time.

The present invention addresses this limitation by providing a single modular kit capable of performing all three functions—extraction, injection, and instrument introduction—while minimizing trauma. The inclusion of RF ablation through the RF needle (10) further enhances safety and treatment options.

### SUMMARY OF THE INVENTION

The Extractor-Injector-Introducer Kit (EIIK) comprises: (a) a steerable RF needle (10) with a curved blunt tip (12) and conductor (14) for coupling to an RF generator; (b) a nested sequence of concentric dilator tubes (20, 22, 24), interlocking to form a guided access tunnel; (c) an extractor tube (30) with a distal cutting element (32) for excising and capturing tissue; and (d) an injector tube (40) including a delivery lumen (42) for the introduction of biological or pharmacological agents.

The outermost dilator tube (24) may remain in place as an introducer sheath, providing access for microsurgical instruments such as aneurysm clips.

### BRIEF DESCRIPTION OF THE DRAWINGS

FIG. 1 illustrates the RF needle (10) with blunt tip (12) and conductor (14).

FIG. 2 illustrates the concentric dilator tubes (20, 22, 24) in exploded view.

FIG. 3 illustrates the extractor tube (30) with distal cutting element (32).

FIG. 4 illustrates the injector tube (40) with delivery lumen (42).

Materials and Tolerances Unless otherwise noted, tolerances are ±0.10 mm linear, ±0.5° angular, and 0.05 mm concentricity. All internal bores are deburred, tips polished to a continuous surface, and components are ultrasonically cleaned and packaged in lint-free poly bags. Preferred material is surgical stainless steel compliant with ISO 7153-1.

FIG. 5 illustrates a schematic flow of the method of use.

### DETAILED DESCRIPTION OF THE INVENTION

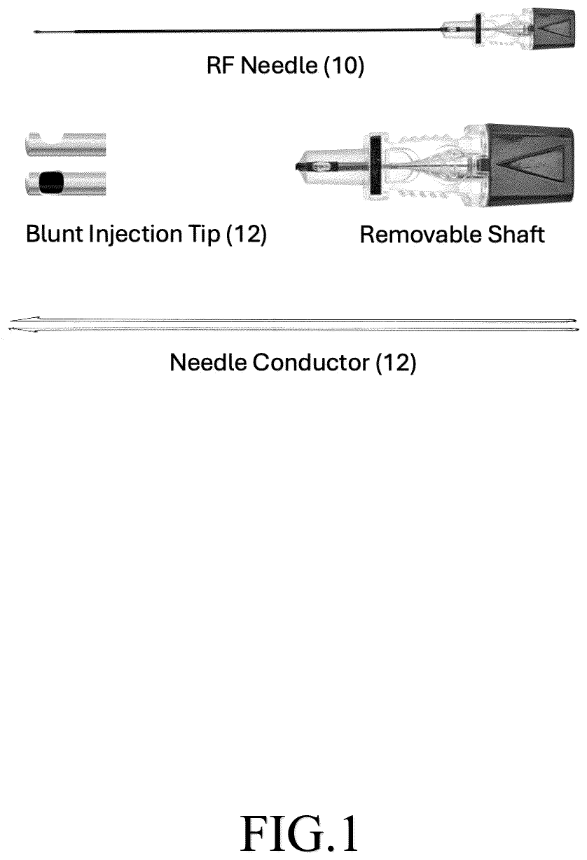

Referring to FIG. 1, the RF needle (10) includes a curved blunt tip (12) to reduce vascular perforation risk and a conductor (14) configured to couple with an RF generator for selective ablation.

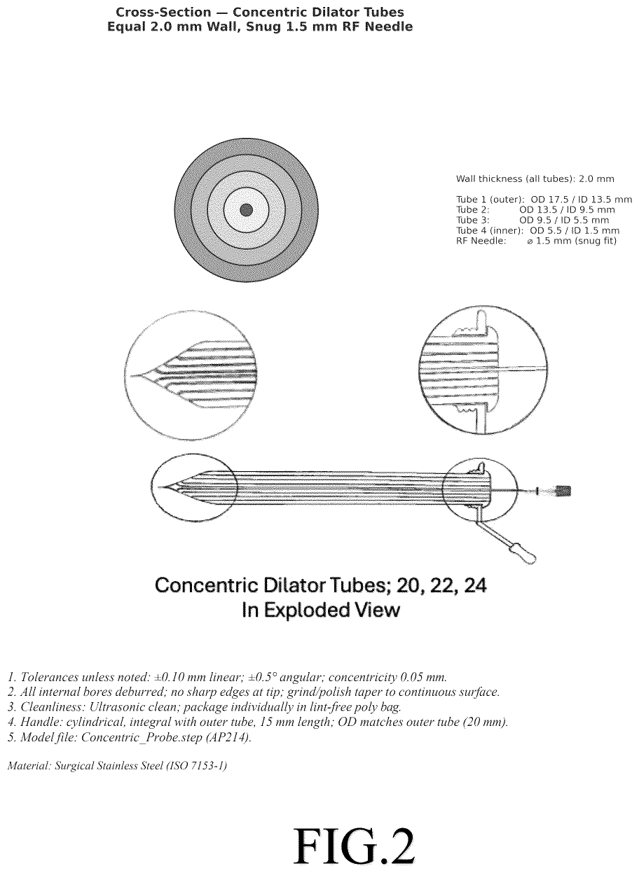

Referring to FIG. 2, the dilator assembly comprises a plurality of concentric dilator tubes (20, 22, 24), each of increasing diameter. These tubes interlock to form a minimally traumatic passage to the lesion site.

Once the dilator assembly is in place, the RF needle (10) and all but the outermost dilator tube (24) are withdrawn. The remaining tube (24) functions as an introducer sheath.

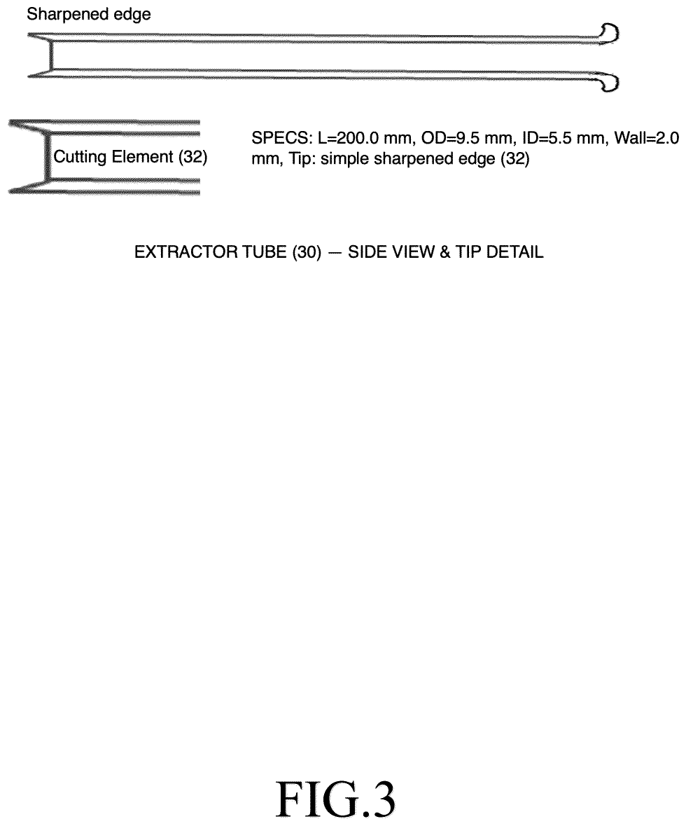

Referring to FIG. 3, the extractor tube (30) includes a distal cutting element (32), which may be a looped wire, blade, or fiber, configured to excise and capture targeted tissue upon rotation.

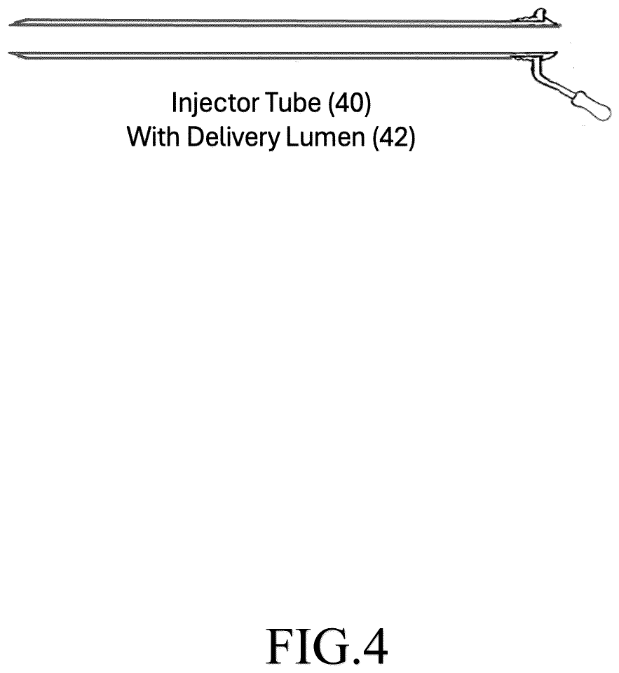

Referring to FIG. 4, the injector tube (40) includes a delivery lumen (42) for introducing therapeutic materials such as stem cells, growth factors, or pharmacological agents to the lesion site.

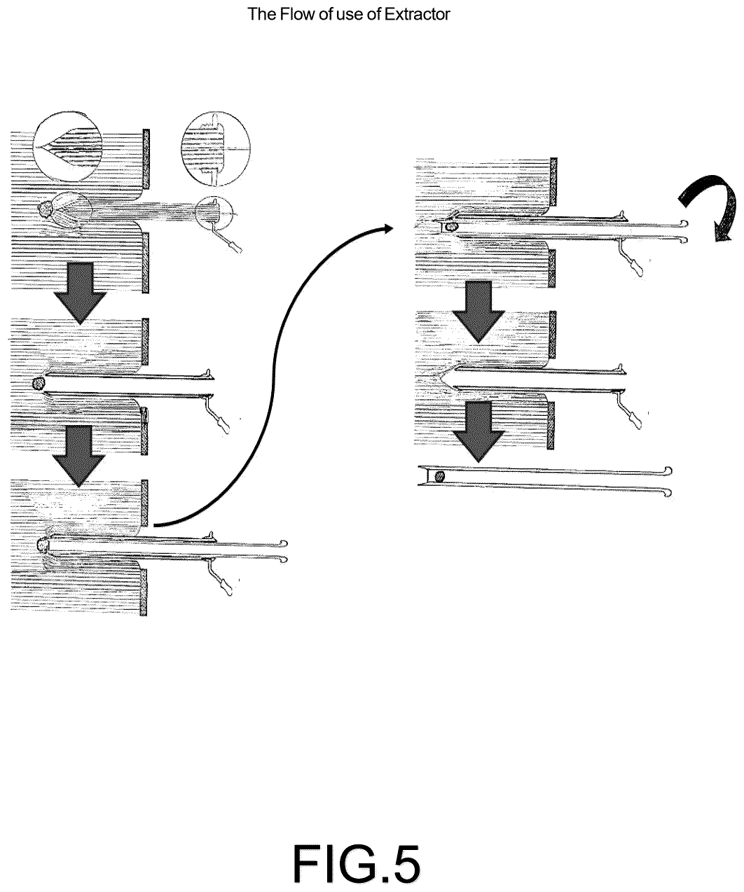

Referring to FIG. 5, in use, the RF needle (10) is inserted under image guidance, optionally energized for ablation. Dilator tubes (20, 22, 24) are sequentially advanced over the RF needle (10). Once positioned, the RF needle (10) and inner dilators (20, 22) are withdrawn, leaving the outermost dilator (24) in place. The extractor tube (30) or injector tube (40) is then advanced through the sheath (24). Upon completion, the devices are withdrawn, and the tissue tract collapses naturally.

1. An extractor-injector-introducer surgical kit (EIIK), comprising: (a) a steerable radiofrequency (RF) needle (10) with a curved, blunt tip (12) configured for safe insertion and optional tissue ablation; (b) a series of concentrically aligned dilator tubes (20, 22, 24) of increasing diameter configured to interlock and guide instruments to a lesion site; (c) an extractor tube (30) configured to be advanced through the dilator tubes, the extractor tube (30) including a distal cutting element (32) for excising and retrieving tissue; and (d) an injector tube (40) configured to be advanced through the dilator tubes for delivering biological or pharmacological material, wherein the outermost dilator tube (24) is configured to remain in place as an introducer sheath.

2. The kit of claim 1, wherein the distal cutting element (32) of the extractor tube (30) comprises a looped wire, fiber, or blade.

3. The kit of claim 1, wherein the injector tube (40) is configured to deliver stem cells, growth factors, or therapeutic drugs.

4. The kit of claim 1, wherein the introducer sheath (24) is dimensioned to accommodate neurosurgical instruments, including aneurysm-clipping devices.

5. A method of extracting, injecting, or introducing instruments to a lesion site, comprising: (a) inserting a steerable RF needle (10) under image guidance to a lesion site; (b) optionally ablating tissue with the RF needle (10); (c) sequentially advancing a plurality of dilator tubes (20, 22, 24) of increasing diameter over the RF needle (10) to form a guided channel; (d) withdrawing the RF needle (10) and all but the outermost dilator tube (24), leaving the outermost tube (24) as an introducer sheath; (e) advancing either an extractor tube (30) to excise and retrieve tissue or an injector tube (40) to deliver therapeutic material; and (f) withdrawing the extractor tube (30), injector tube (40), and introducer sheath (24) to allow natural closure of the tissue tract.
